(19) United States
(12) Patent Application Publication
(10) Pub. No.: US 2026/0250736 A1
(43) Pub. Date: Aug. 27, 2026

(54) SURFACE INTERACTION PROFILES OF BACTERIA AND METHODS FOR THEIR USE

(71) Applicants: Nicholas Konstantine Kotoulas, Toronto (CA); Maria Cynthia Goh, Toronto (CA)

(72) Inventors: Nicholas Konstantine Kotoulas, Toronto (CA); Maria Cynthia Goh, Toronto (CA)

(21) Appl. No.: 19/160,294

(22) PCT Filed: Feb. 27, 2024
(86) PCT No.: PCT/CA2024/050245

§ 371 Date: Aug. 27, 2025

Related U.S. Application Data
(60) Provisional application No. 63/448,374, filed on Feb. 27, 2023.

(51) Int. Cl. C12Q 1/06

(52) U.S. Cl. CPC C12Q 1/06

(57) ABSTRACT

Provided are methods of preparing a premeasurement and/or a surface interaction profile (SIP) for a known type of a bacteria. The methods comprise: providing a defined material; contacting a fluid comprising a known concentration of bacteria with the defined material for a first period of time, thereby forming a contacted material; rinsing the contacted material with a rinsing solution, thereby forming a rinsed contacted material; measuring the bacteria on the rinsed contacted material, thereby generating a measurement; and recording the measurement, thereby preparing a premeasurement for the known type of the bacteria. In some embodiments, methods of identifying a type of a bacteria are provided. In these latter embodiments, the methods comprise similar steps to prepare a measurement and/or SIP as well as a step of comparing the measurement to a premeasurement where a match between the measurement and the premeasurement leads to an identification of the bacteria.

### TECHNICAL FIELD

This invention relates to the identification and/or differentiation of bacteria based on their interactions with defined surfaces.

### BACKGROUND

Advances in rapid precision diagnostics limit the impact of rising antimicrobial resistance by improving antibiotic stewardship (Patel, R. & Fang, F.

C. Diagnostic Stewardship: Opportunity for a Laboratory-Infectious Diseases Partnership. Clin. Infect. Dis. Off. Publ. Infect. Dis. Soc. Am. 67, 799-801 (2018)) and relying less on empirical treatment practices that can lead to increased patient risk (Micek, S. T., Hampton, N. & Kollef, M. Risk Factors and Outcomes for Ineffective Empiric Treatment of Sepsis Caused by Gram-Negative Pathogens: Stratification by Onset of Infection. Antimicrob. Agents Chemother. 62, e01577-17 (2017)). Emerging technologies including broad-range PCR (Tkadlec, J. et al. The use of broad-range bacterial PCR in the diagnosis of infectious diseases: a prospective cohort study. Clin. Microbiol. Infect. Off. Publ. Eur. Soc. Clin. Microbiol. Infect. Dis. 25, 747-752 (2019)), next-generation DNA sequencing (Besser, J., Carleton, H. A., Gerner-Smidt, P., Lindsey, R. L. & Trees, E. Next-generation sequencing technologies and their application to the study and control of bacterial infections. Clin. Microbiol. Infect. 24, 335-341 (2018)), and MALDI-TOF mass spectrometry (Singhal, N., Kumar, M., Kanaujia, P. K. & Virdi, J. S. MALDI-TOF mass spectrometry: an emerging technology for microbial identification and diagnosis. Front. Microbiol. 6, (2015)) largely address issues with lagging detection times while maintaining a high degree of sensitivity and specificity. These techniques have also shown promise in combining onboard bacterial identification with antibiotic susceptibility testing (AST) (Chao, L., Li, J., Zhang, Y., Pu, H. & Yan, X. Application of next generation sequencing-based rapid detection platform for microbiological diagnosis and drug resistance prediction in acute lower respiratory infection. Ann. Trans/. Med. 8, 1644-1644 (2020); Florio, W. et al. Detection of Antibiotic-Resistance by MALDI-TOF Mass Spectrometry: An Expanding Area. Front. Cell. Infect. Microbiol. 10, (2020); and Maxson, T., Blancett, C. D., Graham, A. S., Stefan, C. P. & Minogue, T. D. Rapid antibiotic susceptibility testing from blood culture bottles with species agnostic real-time polymerase chain reaction. PLoS ONE 13, e0209042 (2018)), a sought-after feature in clinical diagnostics. Currently, there is a need to reduce the cost, facility, and specialized training barriers of new technologies in bacterial detection for implementation in low resource settings (Blaschke, A. J. et al. Unmet Diagnostic Needs in Infectious Disease. Diagn. Microbiol. Infect. Dis. 81, 57-59 (2015); and Abou Tayoun, A. N., Burchard, P. R., Malik, I., Scherer, A. & Tsongalis, G. J. Democratizing Molecular Diagnostics for the Developing World. Am. J. Clin. Pathol. 141, 17-24 (2014)). Solutions to combat the global antimicrobial crisis need to be global in nature, necessitating the development of new, rapid, and accessible approaches to bacterial detection.

### SUMMARY

The present invention is based, at least in part, on the development of surface interaction profiles (SIPs) and the use of SIPs to identify types of bacteria.

In illustrative embodiments of the present invention, there is provided a method of preparing a premeasurement for a known type of a bacteria, the method comprising: a) providing a defined material; b) contacting a fluid with the defined material for a first period of time, the fluid comprising the bacteria and having a known concentration of bacteria, thereby forming a contacted material; c) rinsing the contacted material with a rinsing solution by causing the rinsing solution to flow across the contacted material with a first generally consistent flow rate for a second period of time, thereby forming a rinsed contacted material; d) measuring the bacteria on the rinsed contacted material, thereby generating a measurement; and e) recording the measurement, thereby preparing a premeasurement for the known type of the bacteria.

In illustrative embodiments of the present invention, there is provided a method of identifying a type of a bacteria, the method comprising: a) providing a defined material; b) contacting a fluid with the defined material for a first period of time, the fluid comprising the bacteria and having a known concentration of the bacteria, thereby forming a contacted material; c) rinsing the contacted material with a rinsing solution by causing the rinsing solution to flow across the contacted material with a first generally consistent flow rate for a second period of time, thereby forming a rinsed contacted material; d) measuring the bacteria on the rinsed contacted material, thereby generating a measurement; and e) comparing the measurement to a premeasurement, wherein the premeasurement is from a set of premeasurements, the set of premeasurements comprising a plurality of known measurements where each known measurement is a known measurement for one of a plurality of known types of bacteria and the known measurement is obtained using the same flow rates, the same periods of time, and the same defined material and a match between the measurement and the premeasurement identifies the type of the bacteria as the known type of bacteria from the matched premeasurement.

In illustrative embodiments of the present invention, there is provided a method described herein wherein providing the defined material comprises prerinsing the defined material by contacting a prerinsing solution with the defined material.

In illustrative embodiments of the present invention, there is provided a method described herein wherein contacting the prerinsing solution with the defined material comprises dipping the defined material in the prerinsing solution for a third period of time.

In illustrative embodiments of the present invention, there is provided a method described herein wherein contacting the prerinsing solution with the defined material comprises adding the prerinsing solution to the surface of the defined material and allowing the prerinsing solution to sit for a third period of time.

In illustrative embodiments of the present invention, there is provided a method described herein wherein contacting the prerinsing solution with the defined material comprises causing the prerinsing solution to flow across the surface of the defined material with a second generally consistent flow rate for a third period of time.

In illustrative embodiments of the present invention, there is provided a method described herein wherein the second generally consistent flow rate is in a range of from about 0.0 cm/s to about 5 cm/s.

In illustrative embodiments of the present invention, there is provided a method described herein wherein the second generally consistent flow rate is in a range of from about 0 cm/s to about 3 cm/s.

In illustrative embodiments of the present invention, there is provided a method described herein wherein the second generally consistent flow rate is in a range of from about 0.5 cm/s to about 3 cm/s.

In illustrative embodiments of the present invention, there is provided a method described herein wherein the third period of time is not more than 6 hours.

In illustrative embodiments of the present invention, there is provided a method described herein wherein the prerinsing the defined material by contacting a prerinsing solution with the defined material is repeated at least once.

In illustrative embodiments of the present invention, there is provided a method described herein wherein the prerinsing the defined material by contacting a prerinsing solution with the defined material is not repeated.

In illustrative embodiments of the present invention, there is provided a method described herein wherein the rinsing the contacted material with a rinsing solution is repeated at least once.

In illustrative embodiments of the present invention, there is provided a method described herein wherein the rinsing the contacted material with a rinsing solution is not repeated.

In illustrative embodiments of the present invention, there is provided a method described herein wherein the method is repeated one or more times using a different defined material for each of the one or more times.

In illustrative embodiments of the present invention, there is provided a method described herein wherein the different defined material is a substrate upon which a coating is deposited and the coating comprises a material selected from the group consisting of at least one of: a polymer, a polyelectrolyte, a nanoparticle, a surfactant; an organic molecule, a biomolecule, a protein, a poly amino acid, an oligonucleotide, and a nucleic acid.

In illustrative embodiments of the present invention, there is provided a method described herein wherein the coating is deposited in one layer.

In illustrative embodiments of the present invention, there is provided a method described herein wherein the coating is deposited in successive layers to form a multilayer film.

In illustrative embodiments of the present invention, there is provided a method described herein wherein the coating is a PEM.

In illustrative embodiments of the present invention, there is provided a method described herein wherein the different defined material is PDDA, PEI, or PSS.

In illustrative embodiments of the present invention, there is provided a method described herein wherein the defined material is a substrate upon which a coating is deposited and the coating comprises a material selected from the group consisting of at least one of: a polymer, a polyelectrolyte, a nanoparticle, a surfactant; an organic molecule, a biomolecule, a protein, a poly amino acid, an oligonucleotide, and a nucleic acid.

In illustrative embodiments of the present invention, there is provided a method described herein wherein the coating is a PEM.

In illustrative embodiments of the present invention, there is provided a method described herein wherein the defined material is PDDA, PEI, or PSS.

In illustrative embodiments of the present invention, there is provided a method described herein wherein the first generally consistent flow rate is in a range of from about 0.5 cm/s to about 5 cm/s.

In illustrative embodiments of the present invention, there is provided a method described herein wherein the first generally consistent flow rate is in a range of from about 1 cm/s to about 3 cm/s.

In illustrative embodiments of the present invention, there is provided a method described herein wherein the first generally consistent flow rate is in a range of from about 2.5 cm/s to about 3 cm/s.

In illustrative embodiments of the present invention, there is provided a method described herein wherein the first period of time is in a range of from between about 1 minute to about 20 minutes.

In illustrative embodiments of the present invention, there is provided a method described herein wherein the first period of time is in a range of from about 1 minute to about 10 minutes.

In illustrative embodiments of the present invention, there is provided a method described herein wherein the second period of time is not less than 1 minute.

In illustrative embodiments of the present invention, there is provided a method described herein wherein the second period of time is not more than 10 minutes.

In illustrative embodiments of the present invention, there is provided a method described herein wherein the contacting the fluid comprising the bacteria with the defined material for a first period of time, comprises resting the fluid comprising the bacteria on a top surface of the defined material for the first period of time.

In illustrative embodiments of the present invention, there is provided a method described herein wherein the contacting the fluid comprising the bacteria with the defined material for a first period of time, comprises causing the fluid comprising the bacteria to flow across the defined material with a third generally consistent flow rate for the first period of time.

In illustrative embodiments of the present invention, there is provided a method described herein wherein the third generally consistent flow rate is in a range of from about 0.05 cm/s to about 1 cm/s.

In illustrative embodiments of the present invention, there is provided a method described herein wherein the set of premeasurements consists of a subset of premeasurements wherein the subset of premeasurements is limited to premeasurements of known types of bacteria of interest.

In illustrative embodiments of the present invention, there is provided a method described herein wherein the measurement and each premeasurement is expressed as a member selected from the group consisting of: bacterial count per unit area, intensity of light transmitted through, intensity of light reflected, intensity of light diffracted, and intensity of light scattered.

In illustrative embodiments of the present invention, there is provided a method described herein wherein the measurement is expressed as a bacterial count per unit area measurement and each premeasurement is expressed as a bacterial count per unit area premeasurement.

In illustrative embodiments of the present invention, there is provided a method described herein wherein the premeasurement is expressed as an average obtained from averaging a plurality of premeasurements.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1 Experimental design and surface interaction profiles (SIPs) of all test bacteria. a, An illustration of a polyelectrolyte multilayer film assembled via the layer-by-layer approach. b-d, The chemical structures of polydiallyldimethylammonium chloride (PDDA), polystyrene sulfonate (PSS), and polyethylenamine (PEI). e, A schematic of six test PEM surfaces (circles) encased in microfluidic channels (squares) in a polystyrene Petri dish. f, The protocol for bacterial-surface interactions: (1) pre-soaking PEM with PBS buffer (30 min), (2) introducing bacterial suspensions to each channel, (3) leaving them in contact for 10 minutes, and (4) rinsing channels with PBS. g, A schematic depicting the SIP, a novel bacterial identifier produced by combining interaction strengths between a given bacterium and a series of chemically unique surfaces. h, The interaction strengths of all twelve test bacteria plotted as a heatmap for SIP comparison. i, The ESKAPE pathogen SIPs organized by Gram stain (mean±SEM). j-m, All SIPs across S. aureus mutants (mean±SEM).

FIG. 2 Attachment curves of all test bacteria. All plots of cell counts per PDDA-(thick black), PEI-(dashed), and PSS-topped (thin black) PEM surfaces per cell type (N=15 test bacteria, N=540 PEMs) (mean±SEM): a-c Gram-negative (−) ESKAPE pathogens, d-f Gram-positive (+) ESKAPE pathogens, g-k S. aureus SNPs, I MRSA ΔtarO, and m-o blind test pathogens (i: E. faecalis, ii: A. baumannii, and iii: K. pneumoniae).

FIG. 3 Blind experiment SIP matching. a, All ESKAPE and blind pathogen SIPs plotted as a heat map. b, The bacterial-surface attachment curves of blind test pathogens i, ii, and iii compared with their correctly identified ESKAPE counterparts E. faecalis, A. baumannii, and K. pneumoniae, respectively (mean±SEM).

FIG. 4 PEM template and fluid chip assembly. a, A Petri dish lid template outlining the relative positions and dimensions of marked circular boundaries used to build each PEM. b, The location, dimensions, and order of double-sided tape addition. c, A schematic displaying the location of added lane toppers (glass coverslips) and lane intlet hydrophobic markings.

FIG. 5 PEM assembly rinsing protocol for unidirectional flow. a, The Petri lid was kept at a ~35° angle during rinsing. b, Surfaces were first lightly rinsed in sequence from left to right (1-3). c, The chip was rotated by 180° while maintaining the aforementioned pitch angle. d, The next three surfaces were lightly rinsed in sequence from left to right (4-6). e, The disposal of collected aqueous waste. f, The Petri lid was inverted and tapped (8×) on a lab bench before adding the next polyelectrolyte solution.

FIG. 6 Introducing bacterial suspensions to the fluid lanes. An illustration of the protocol used to introduce each concentration of bacterial suspension to an individual fluid lane. a, Fluid lanes containing PEM surfaces were pre-wet with standard concentration PBS buffer. b, The bacterial suspension was introduced to the fluid lane via wicking. c, The bacterial suspension was left to interact with the PEM surface for 10 minutes. d, The fluid lane was rinsed with PBS buffer. e, The surface was imaged using an inverted microscope.

### DETAILED DESCRIPTION

The present invention is based, at least in part, on the development of surface interaction profiles (SIPs) and the use of SIPs to identify types of bacteria. Depending on the context, as used herein, the term “SIP” may refer to a “measurement” and/or a “premeasurement” or alternatively reference to a “measurement” and/or a “premeasurement” may refer to a SIP, though the context will make this clear for each use of each term. In essence, each of “SIP”, “premeasurement”, and “measurement” refer to the profile of a type of bacteria and its interaction with a particular defined surface, but depending on the time and manner that the profile is being used, the term “measurement” and/or “premeasurement” may be helpful to understand the context of the profile. Typically, a “measurement”, when referring to a SIP, is meant to indicate that the profile has been recently obtained (temporally meaning closer to now) using a method of the present invention. Further, a “premeasurement”, when referring to a SIP, is generally meant to indicate that the profile has been generated using a first instance of a method of the present invention prior to carrying out a second instance of the method of the present invention. The “premeasurement” is often being used as a basis for comparison to a “measurement”. In some cases a SIP, premeasurement and/or a measurement is expressed as number, a curve, a slope or other mathematical representation of the pattern of bacteria on the defined surface. A pattern of bacteria, for example and without limitation, may be a concentration of bacteria on the surface, a surface bound concentration of bacteria, density of cells on the surface, or any combination thereof.

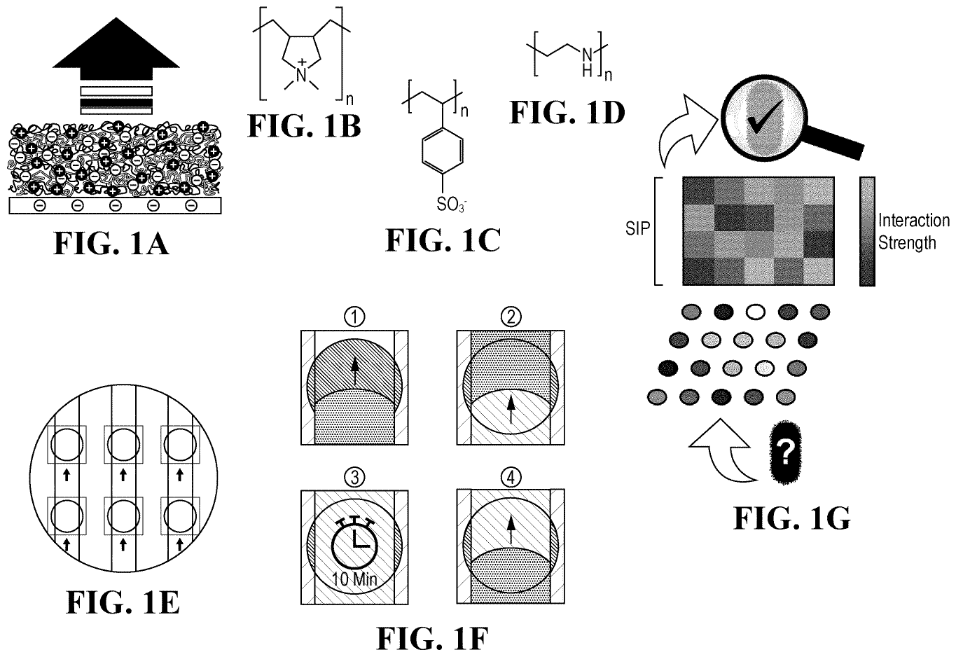

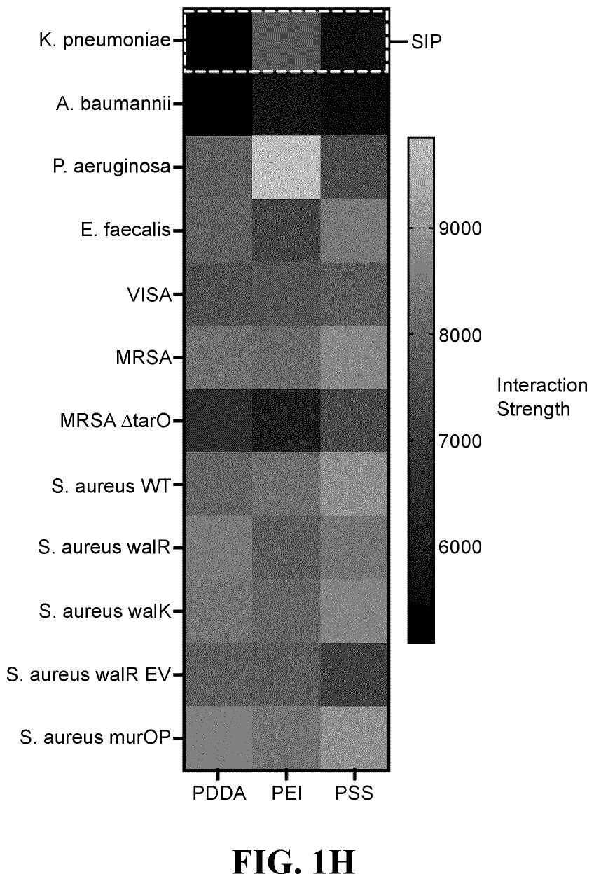

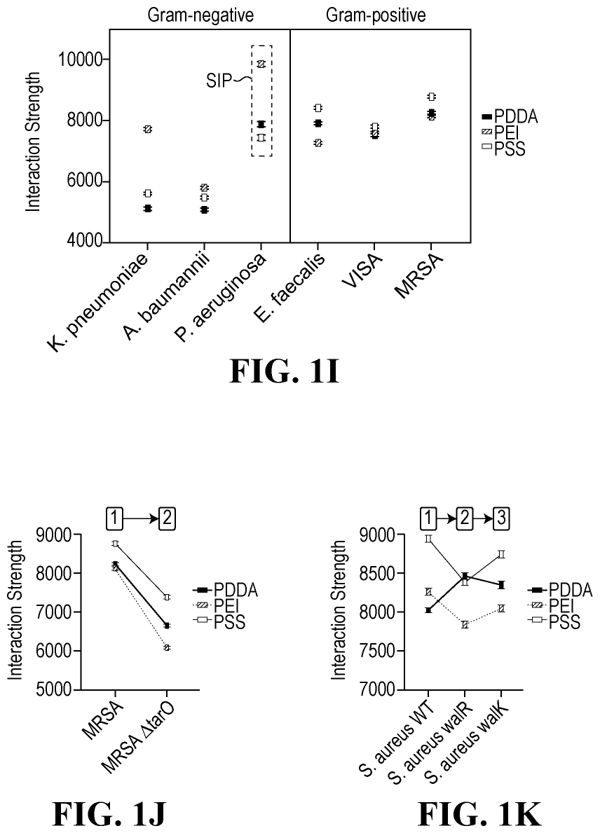

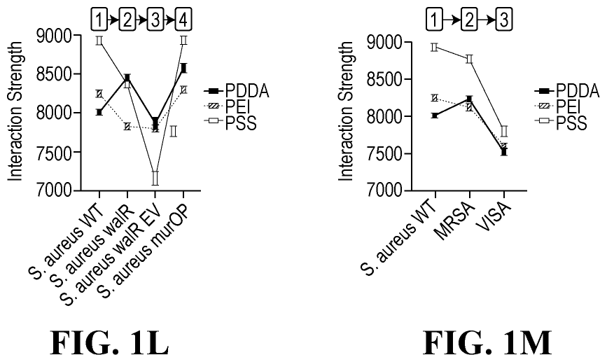

The use of SIPs, as described herein, is able to differentiate between bacterial species as well as bacteria of the same species having only differences in phenotype on the basis of small differences in their genomes. The present invention provides methods capable of differentiating and identifying bacteria by their uniquely complex exteriors. Methods of the present invention expose a type of bacterium to a surface and/or to a set of surfaces with distinct chemical and physical properties and combine each individual cell-surface interaction strength into a fingerprint-like identifier, often termed herein as a Surface Interaction Profile (SIP) or as a “premeasurement” or as a “measurement” (FIG. 1 g). In experiments described herein, the use of SIPs in distinguishing bacteria more broadly by Gram stain and by individual species via testing a set of hospital-acquired ESKAPE pathogens as well as to distinguish between a series of Staphylococcus aureus (S. aureus) strains with single point mutations in their genome affecting both cell wall thickness and antibiotic resistance are achieved.

Numerous isoforms of MRSA, including VISA, are common risk factors in clinical settings and present a heightened threat to surgical patients due to potential surgical site infections (SSIs) (Roe, C. C. et al. Whole genome SNP typing to investigate methicillin-resistant Staphylococcus aureus carriage in a health-care provider as the source of multiple surgical site infections. Hereditas 153, 11 (2016)). Having a tool to easily distinguish between these SNPs would assist in identifying antibiotic susceptibilities and in tracking the source of these nosocomial outbreaks to reduce the impact of SSIs and other complications.

SIPs can be used to identify cells by Gram stain or differentiate by individual species, SIPs are also sensitive enough to distinguish between SNPs. Differentiating antibiotic-resistant isoforms using SIPs to screen for other high-risk resistant nosocomial pathogens is clinically relevant as a rapid-capable tool that combines both identification of species and antibiotic-resistant isoforms in a single, low-cost test.

In illustrative embodiments of the present invention, there is provided a method of preparing a premeasurement and/or SIP for a known type of a bacteria. The method comprises: a) providing a defined material; b) contacting a fluid comprising the known type of bacteria with the defined material, thereby forming a contacted material; c) rinsing the contacted material with a rinsing solution, thereby forming a rinsed contacted material; d) measuring the bacteria on the rinsed contacted material; and e) recording the measurement, thereby preparing a premeasurement and/or SIP for the known type of the bacteria.

As used herein, the “defined material” is a material that has a reproducible surface. The defined material may be a single material or may be a material that is deposited on a substrate or may be a complex of multiple different materials. As used herein, a “reproducible surface” is a surface that has a consistent and reproducible structure having distinct and consistent chemical and physical properties. As used herein, the phrase “chemical and physical properties” is used interchangeably with the phrase “chemicophysical properties” as well as the phrase “physiochemical properties” and the meaning of each of these three phrases are substantively the same. A reproducible surface is often obtained by preparing the surface under stringent conditions, which conditions are designed to reduce or eliminate variations in the final surface. For example, preparing a surface using the same defined material precursors on a different substrate may cause differences in the chemical and physical properties of the final surfaces. Further, rinsing steps used in preparation of the defined material, if not conducted appropriately, can also result in differences in some surfaces of a defined material. Such defined materials are known to a person of skill in the art and methods of preparing and making such defined materials are also well understood by the person of skill in the art (see, for example, Petrila, L.-M., Bucatariu, F., Mihai, M. & Teodosiu, C. Polyelectrolyte Multilayers: An Overview on Fabrication, Properties, and Biomedical and Environmental Applications. Materials 14, 4152 (2021); and Coulter, M. M., Dos Santos, D. S., Loo, R. W. & Goh, M. C. Combinatorial polyelectrolyte multilayer film fabrication. J. Nanosci. Nanotechnol. 9, 6421-6426 (2009)). Examples of defined materials, without limitation, that may be used in methods of the present invention include, but are not limited to, a coating deposited on a substrate, polyelectrolyte multilayers (PEMs), polymers; polyelectrolytes; nanoparticles; surfactants; organic molecules; biomolecules (protein, poly amino acids, oligonucleotides, nucleic acids), deposited in one layer, or in successive layers to form a multilayer film. Examples of defined materials, without limitation that may be used in methods of the present invention include, but are not limited to, the strongly positive polydiallyldimethylammonium chloride (PDDA) (FIG. 1 b), the strongly negative polystyrene sulfonate (PSS) (FIG. 1 c), and the weakly positive polyethylenimine (PEI) (FIG. 1 d).

The use of defined materials in methods of the present invention relates to the complexity of the outer surfaces of bacteria. Provided that the surface provides a generally consistent interaction (via the chemical and physical properties of the surface), the bacteria will interact with such a surface in a generally consistent manner that is based on the complexity of the outer surface of the bacteria. For example, bacteria with different surface charges will respond differently to the same defined material having a particular charge. Further, a protein present in one type of bacteria may interact strongly with a particular defined material while another type of bacteria lacking this protein may not interact with the same particular defined material. The absence or presence of a protein may be due only to a single point mutation in the entire genome of the bacteria, making the methods of the present invention quite sensitive.

Without being bound by theory, it is also thought that if the defined material provides a more complex interaction with the bacteria, the more sensitive the methods of the present invention will be. PEMs provide a range of adjustable surface properties which are dictated by the conditions for their deposition and the types of polymers added, primarily in the topmost exposed layer (see for example, Petrila, L.-M., Bucatariu, F., Mihai, M. & Teodosiu, C. Polyelectrolyte Multilayers: An Overview on Fabrication, Properties, and Biomedical and Environmental Applications. Materials 14, 4152 (2021) and Coulter, M. M., Dos Santos, D. S., Loo, R. W. & Goh, M. C. Combinatorial polyelectrolyte multilayer film fabrication. J. Nanosci. Nanotechnol. 9, 6421-6426 (2009)). To detect subtleties in the chemical and physical properties of a complex surface, such as the surface of a bacteria, the probe itself (e.g. the defined material), should match in its compositional complexity. At the scale of interaction between bacterial surface moieties and defined materials, such as PEM films, the constituents of the defined material (e.g. polymeric constituents of a PEM) also appear complex and heterogeneous. At the molecular level, defined materials (e.g. PEM polymer films) contain a repeating patchwork of both charged and uncharged as well as polar and non-polar domains, unique from constituent to constituent (e.g. polymer to polymer in the case of PEMs (FIGS. 1 b-d)), and therefore produce a diverse range of possible interactions. It is described herein that an array of constituents in a defined material used as a single probe (e.g. PEMs) rivals the complexity of the exterior of a bacterium, enabling the use of the entire cell exterior as a determinant feature rather than seeking a single molecular target to differentiate.

Further, if multiple SIPs and/or premeasurements and/or measurements for a single particular defined material for single known type of bacteria are used to prepare an average SIP and/or premeasurement and/or measurement, then it is believed that the methods of the present invention will be more sensitive and/or accurate. Further still, if multiple different defined materials are used to generate multiple SIPs and/or premeasurements and/or measurements for a single known type of bacteria, then it is believed that the methods of the present invention will be more sensitive and/or accurate.

The geometry of the defined material is also relevant to the generation of a SIP. While the exact geometry of the defined material is not of particular importance, using defined materials of a similar, and preferably the same, geometry is preferred for generation of consistent and comparable SIPs. For example, changing a length or a width of the defined material may influence the flow rate of fluids across the surface of the defined material and use of consistent flow rates are desirable. Further, the surface area of the defined material could be as large as is practical, but it should not be smaller than the way it is probed (e.g., if optical microscopy is used to probe, then the area should be at least a few times the field of view so as to enable multiple images; if a laser beam is used to probe, then the surface are of the defined material should be at least larger than the laser beam diameter). Further still, the thickness of the defined material could be relevant to the passage of electromagnetic waves through the material and hence the thickness of the defined material may also have an impact on the generation of SIPs depending on the type of probe used for measuring the SIP. Using defined materials having similar or the same geometries is preferred for the generation of consistent and comparable SIPs, premeasurements and/or measurements.

The fluid comprising the bacteria has a known concentration of bacteria. The exact concentration of the bacteria in the fluid is not particularly important for success of the methods of the present invention. However, the concentration of the bacteria in the fluid may result in a particular SIP and/or premeasurement and/or measurement being obtained and it is preferred to compare SIPs and/or premeasurements and/or measurements that have been generated using fluids having the same concentration of bacteria. Further, it is preferred that the properties of the fluid comprising the bacteria are also the same. For example, using a fluid that has many different components that interact with the defined surface as compared to a fluid that has fewer components that interact with the defined surface may result in the generation of different SIPs and/or premeasurements and/or measurements. When comparing a premeasurement to a measurement, it is preferred that a similar fluid comprising the bacteria, and ideally the same fluid comprising the bacteria, is used to generate the measurement as was used to generate the premeasurement.

The manner of contact between the fluid comprising the bacteria and the defined material should also be consistent. In some illustrative embodiments of the present invention, the contact between the fluid comprising the bacteria and the defined material is achieved by introducing the solution to the defined material, letting the fluid comprising the bacteria sit for a first period of time and then removing the fluid comprising the bacteria from the defined material. The fluid comprising the bacteria may be removed, for example and without limitation, by wicking with an absorbent material, or careful removal using an instrument such as a pipette, or by causing a rinsing solution to displace the fluid comprising the bacteria. In other illustrative embodiments, the fluid comprising the bacteria is introduced in a third generally consistent flow rate across the defined material such that the bacteria has ample time to interact with the defined material. In these latter embodiments, it is preferred that the third generally consistent flow rate is slow and often in these latter embodiments the surface of the defined material contacted with the fluid comprising the bacteria is relatively large so that the slow third generally consistent flow rate does not prevent the bacteria from adequately contacting the material. A slow flow rate reduces disruptions to the fluid comprising the bacteria which may reduce settling of the bacteria towards the surface. In all of these embodiments, any flow that is used with respect to the fluid comprising the bacteria, including flow induced via wicking, or flow induced from the use of an instrument, or flow induced by displacement with a rinsing solution, or simply the flow of the fluid comprising the bacteria across the surface of the defined material, should avoid inducing shear stresses that significantly remove the bacteria from the defined material. In some illustrative embodiments, the second generally consistent flow rate is in a range of from about 0.0 cm/s to about 5 cm/s. In some other illustrated embodiments, the second generally consistent flow rate is in a range of from about 0.0 cm/s to about 3 cm/s. In still other illustrative embodiments the second generally consistent flow rate is in a range of from about 0.0 cm/s to about 0.5 cm/s.

By causing the fluid comprising the bacteria to contact the defined material with a consistent protocol, a consistent SIP and/or premeasurement may be obtained. Here again, the manner of contact and the particular first period of time is not of particular importance, but comparing SIPs generated using the same manner of contact and the same first periods of time will lead to more accuracy when using methods of the present invention.

If the fluid comprising the bacteria is contacted with the defined material using consistent parameters, then consistent SIPs and/or premeasurements may be obtained. Such techniques result in generation of a consistent contacted material.

The use of a rinsing solution to flow across the contacted material in methods of the present invention leads to greater consistency. Rinsing steps in methods of the present invention may be conducted for a single time in a single rinsing step or may be repeated using two or more rinsing steps concurrently, prior to carrying out the next steps. A rinsing solution is a solution that is more easily made homogeneous in its makeup than the fluid comprising a known concentration of bacteria. This is in part due to the nature of the bacterial metabolism leading to a more dynamic composition of the fluid comprising the bacteria. By causing the rinsing solution to flow across the contacted material with a first generally consistent flow rate for a second period of time, more consistent SIPs and/or premeasurements and/or measurements may be obtained. Here again, the use of the same type of rinsing solution, for the same periods of time, and for the same flow rates is preferred in order to prepare comparable SIPs and/or premeasurements and/or measurements. By using consistent parameters for rinsing, the SIPs and/or premeasurements and/or measurements obtained are more consistent and such techniques result in generation of a consistent rinsed contacted material. Rinsing solutions suitable for use in the present invention include, but are not limited to, salt solutions, phosphate buffer saline, saline, isotonic solutions, water, deionized water, distilled water and combinations thereof.

In some illustrative embodiments, there is provided a method of identifying a type of a bacteria. Such methods comprise: a) providing a defined material; b) contacting a fluid comprising the bacteria with the defined material, thereby forming a contacted material; c) rinsing the contacted material with a rinsing solution, thereby forming a rinsed contacted material; d) measuring the bacteria on the rinsed contacted material, thereby generating a measurement; and e) comparing the measurement to a premeasurement wherein the premeasurement is from a set of premeasurements, the set of premeasurements comprising a plurality of known measurements where each known measurement is a known measurement for one of a plurality of known types of bacteria and the known measurement is obtained using the same flow rates, the same periods of time, and the same defined material and a match between the measurement and the premeasurement identifies the type of the bacteria as the known type of bacteria from the matched premeasurement.

In these illustrative embodiments, steps a) through d) are identical in nature to steps a) through d) of methods used to generate a premeasurement and the above description also applies to these steps, except that the type of bacteria in the fluid comprising the bacteria in these steps is unknown. Step e) of these embodiments is, in essence, the comparing of the measurement obtained from steps a) to d) using a fluid comprising an unknown type of bacteria to a premeasurement obtained earlier using a known type of bacteria. If the comparison of the measurement to the premeasurement matches on the basis that they are similar or the same, then this is an indication that the type of bacteria used to generate the measurement is the same type of bacteria that was used to generate the premeasurement. If the comparison does not match, then this is an indication that the type of bacteria is different. The measurement obtained may be compared to more than one premeasurement in order to rule out many different types of bacteria and/or to try to identify a match.

In some embodiments of the present invention, it is preferred to prerinse the defined material by causing a prerinsing solution to flow across the material with a second generally consistent flow rate for a third period of time. Prerinsing steps in methods of the present invention may be conducted for a single time in a single prerinsing step or may be repeated using two or more prerinsing steps concurrently, prior to carrying out the next steps. Prerinsing of the defined material provides for an added level of consistency leading to more sensitivity and/or accuracy. It is not required that a prerinsing step be carried out for both a measurement and a premeasurement for adequate comparison, but it is preferred that for both generation of the measurement and generation of the premeasurements that are to be used for comparison the method used is similar and preferably the same.

Prerinsing of the defined material provides for the defined material to be wetted by the prerinsing solution. It is possible to provide the defined material in a prewetted state, but wetting soon before introduction of the fluid comprising the bacteria provides a more consistent wetted and/or relaxed surface of the defined material. A wetted and/or relaxed surface of the defined material provides an opportunity for the constituents of the defined materials to conform to a wet environment prior to the introduction of the bacteria. For example, polymers and/or proteins may rearrange their conformation when in contact with a solution. Having polymers in a consistent conformation is beneficial to sensitivity and/or accuracy of the methods of the present invention. If the defined material is not wet, it may take time for the polymers to relax once exposed to the wet environment of the fluid comprising the bacteria, which may lead to the surface of the defined material evolving with time as the bacteria begin to interact with the surface of the defined material. In some illustrative embodiments, the prerinsing solution is contacted with the defined material by causing the prerinsing solution to flow across the surface of the defined material with a second generally consistent flow rate for a third period of time. In some other illustrative embodiments, the surface of the defined material is wetted by dipping it in solution. In some other illustrative embodiments, a prerinsing solution may be added to the surface of the defined material and allowed to sit for a third period of time in order to provide a wetted defined material and in these embodiments the second generally consistent flow rate is 0 cm/s.

In general, the third period of time, which third period of time is the time period for prerinsing when the defined material is prepared using a prerinsing step, is dependent on the makeup of the defined material. Often polymer relaxation time is minutes to hours, depending on the kind of polymer, its chain length, the solution conditions, temperature, etc. Alternatively, if the coating is a smaller molecule, like a surfactant or even short chains, almost no relaxation time may be needed and hence the third period of time could be a matter of seconds or a fraction of a second. In some preferred embodiments, the third period of time is not more than about 6 hours. In some other preferred embodiments, the third period of time is not less than 10 minutes. In some other preferred embodiments, the third period of time is between about 15 minutes and about 40 minutes. In other preferred embodiments still, the third period of time is about 30 minutes.

Suitable flow rates for use for the first generally consistent first flow rate, which is the flow rate for rinsing the contacted material with the rinsing solution, include any flow rate as long as it is consistent between SIPs generated that are to be compared. In some illustrative embodiments, the first generally consistent flow rate is in a range of from about 0.5 cm/s to about 5 cm/s. In other illustrative embodiments, the first generally consistent flow rate is in a range of from about 1 cm/s to about 3 cm/s. In still other illustrative embodiments, the first generally consistent flow rate is in a range of from about 2.5 cm/s to about 3 cm/s.

Suitable flow rates for use for the second generally consistent first flow rate, which is the flow rate for contacting the prerinsing solution with the defined material by causing the prerinsing solution to flow across the surface of the defined material, include any flow rate as long as it is consistent between SIPs generated that are to be compared. In some illustrative embodiments, the second generally consistent flow rate is in a range of from about 0.0 cm/s to about 5 cm/s. In some other illustrated embodiments, the second generally consistent flow rate is in a range of from about 0 cm/s to about 3 cm/s. In still other illustrative embodiments the second generally consistent flow rate is in a range of from about 0.5 cm/s to about 3 cm/s.

Use of the term “generally consistent” refers to the fact that some deviation in the flow rate is permissible as long as the flow rate is within about 10% of the nominal flow rate throughout the duration of the period of time. In some preferred embodiments, “generally consistent” refers to a flow rate that deviates from the nominal flow rate by only about 5%. In some other preferred embodiments, “generally consistent” refers to a flow rate that deviates from the nominal flow rate by only about 2%. In some other preferred embodiments, “generally consistent” refers to a flow rate that deviates from the nominal flow rate by only about 1%.

The first period of time for which the fluid comprising bacteria is contacted with the defined material relates to allowing the bacteria opportunity to interact with the surface. The length of contact the bacteria has with the surface must be long enough to allow the bacteria to interact with the surface, but must also not be too long to prevent the bacteria from growing pods. In other words, the bacteria, if left to contact with surfaces too long will eventually grow and attach themselves to the surface in pods and/or colonies. Such growth is to be avoided so that the only means of attachment by the bacteria to the surface of the defined material are the surface interactions between the bacteria and the surface of the defined material and not the response of the bacterial growth. The exact length of this period of time is in part dependent on the geometry of the defined material as well as the bacterial species itself. Often the first period of time is not less than about 1 minute and not more than about 20 minutes. In some preferred embodiments the first period of time is not less than about 5 minutes and not more than about 15 minutes. In some other preferred embodiments, the first period of time is about 10 minutes.

Suitable periods of time for the second time period, which second time period is the time period for which the contacted material is rinsed, thereby forming a rinsed contacted material, are times not less than about 1 minute. In some preferred embodiments, the second period of time is not less than about 5 minutes. In some other preferred embodiments, the second period of time is not less than about 10 minutes. In some other preferred embodiments, the second period of time is not more than about 10 minutes.

In illustrative embodiments, methods of the present invention may be repeated one or more times using a different defined material for each of the one or more times. When repeating methods of the present invention in the manner, it is preferred that only the defined material is different and that the other parameters, such as flow rates, periods of time, concentrations, etc. remain generally consistent and/or the same. In other words, a unique SIP will be generated for each type of bacteria on each defined material. By comparing more than one measurement, each measurement obtained for the same type of bacteria on a different material, multiple comparisons of SIPs (i.e. measurements to premeasurements) may be made to increase the accuracy and sensitivity of the identification of types of bacteria using methods of the present invention.

Methods of the present invention may also be used to quickly identify a bacteria of particular interest, for example an antibiotic resistant strain of a bacteria thought to be the cause of an infection. In such embodiments of the present invention, it is helpful to compare a measurement to a particular subset of premeasurements which is limited to premeasurements of known types of bacteria of interest. By doing this, identification of the type of bacteria may be obtained more quickly. Alternatively, a particular type of bacteria, for example a bacteria that is of critical clinical relevance, may be ruled out quickly in the event of no match between SIPs.

A SIP may be measured and expressed in any one of a number of ways and any one of these ways is a suitable way to express a SIP provided that the comparison of SIPs is conducted using SIPs having been obtained using the same measuring techniques and expressions thereof. SIPs may be measured using microscopy techniques, light and/or other electromagnetic wave reflection, scattering, and/or intensity measuring techniques. Further, mathematical manipulation of the expression of the measuring, for example averaging, or preparation of a curve, etc. may also form part of the measuring technique. Such measuring techniques and mathematical manipulations thereof are known and understood to the person of skill in the art. One preferred way to express a SIP is to use an optical microscope and count the number of bacteria in a defined area on the surface. This count may be expressed as the SIP for that type of bacteria on that defined material. Alternatively, the SIP could be expressed as an optical density, or using light, expressing a SIP as an intensity change either in reflectance or diffraction. Preferred measures and expression of SIPs include, but are not limited to bacterial count per unit area, intensity of light transmitted through, intensity of light reflected, and/or intensity of light scattered.

SIPs, when expressed as a measurement, will have normal experimental variation within those measurements. In order to refine a SIP measurement, it is helpful to generate a premeasurement by conducting a method of the present invention multiple times to obtain a set of premeasurements for a particular known type of bacteria and then to average that set of premeasurements to generate a single averaged premeasurement of that particular known type of bacteria. In such cases, the premeasurement is an average obtained from averaging a plurality of premeasurements for the particular, known type of bacteria. Similarly, a measurement of a type of bacteria to be identified may also be generated using an average obtained from multiple iterations of methods of the present invention, where the multiple iterations each use generally consistent and/or the same protocol including the materials and the procedures, such as defined material, periods of time, concentrations, flow rates, etc. It is also possible to use other mathematically applicable methods of combining multiple results into a single result and/or single expression of multiple results, such as preparing slopes and/or curves, besides averaging. Such techniques are known to the person of skill in the art.

### EXAMPLES

The following examples are illustrative of some of the embodiments of the invention described herein. These examples do not limit the spirit or scope of the invention in any way.

### Methods and Materials

The following methods and materials were used in the Examples that follow the Methods and Materials sections.

### Polymer Stock Solutions

The stock solutions (10 mg/mL) for PDDA (Aldrich, 208.00 mg/mL), PEI (Aldrich, 50% wt), and PSS (Aldrich, 344.13 mg/mL) polyelectrolytes were prepared by weight and dissolved in deionized (DI) water (pH 6.998, Millipore MilliQ) with magnetic stirring. Three working polymer solutions (1 mg/mL) for each polymer type were prepared by diluting each respective stock (×10) with DI water.

Building PEMs through layer-by-layer assembly

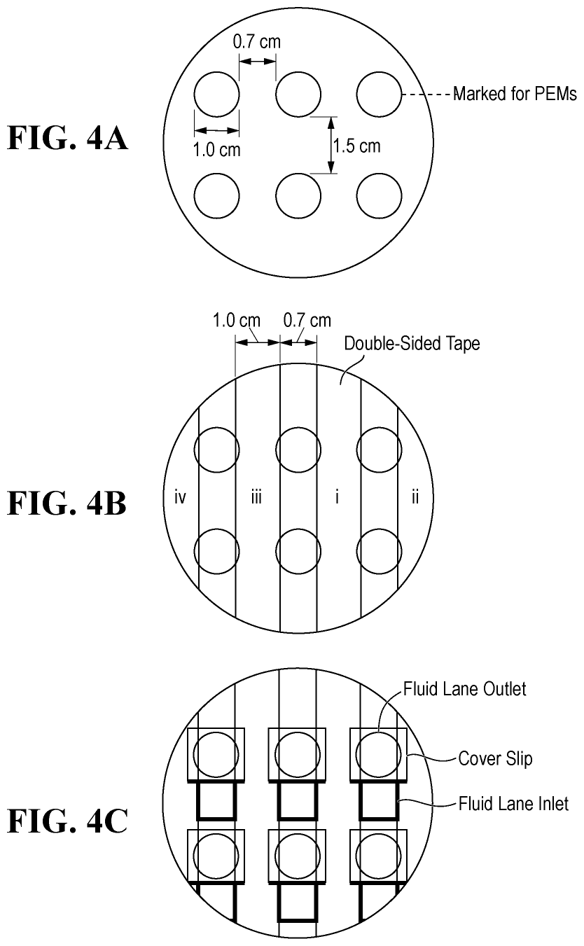

Using a template (FIG. 4 a), thirty-six small circles (1 cm diameter) were drawn across the interior of six Petri dish lids that formed the perimeter of each individual PEM surface. Three types of PEMs were prepared with the following alternating sequence of positive and negative polymer layers: the 5.5 bilayer PDDA-topped (+) PEM (PDDA-PSS-PDDA-PSS-PDDA-PSS-PDDA-PSS-PDDA-PSS-PDDA), the 5.5 bilayer PEI-topped (+) PEM (PEI-PSS-PEI-PSS-PEI-PSS-PEI-PSS-PEI-PSS-PEI), and the 5 bilayer PSS-topped (−) PEM (PDDA-PSS-PDDA-PSS-PDDA-PSS-PDDA-PSS-PDDA-PSS).

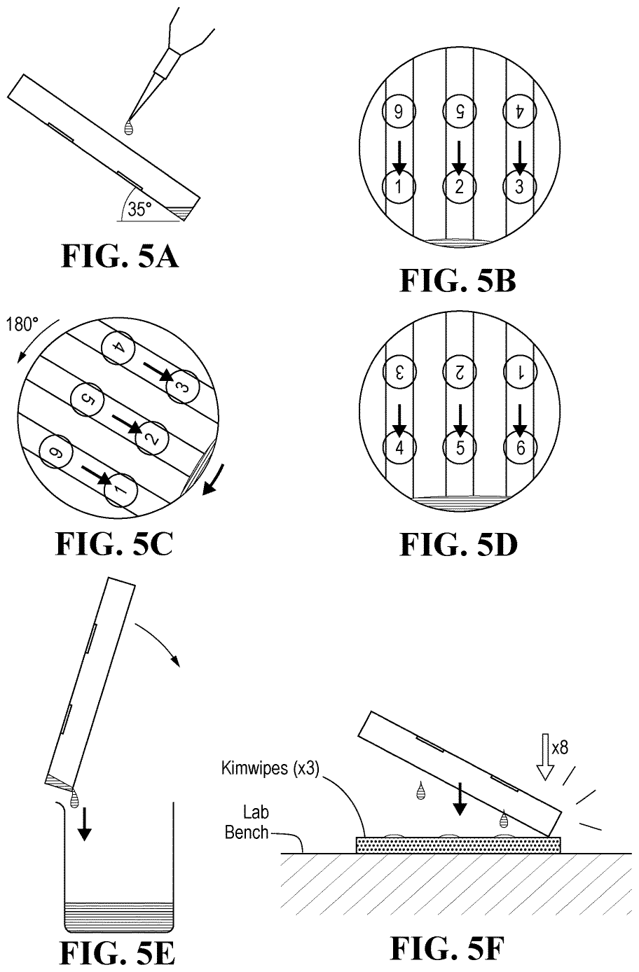

To begin, a small aliquot (100 μL) of positively charged polymer was added to each marked circle: PDDA (1 mg/mL) for PDDA-(N=12) and PSS-topped (N=12) PEMs, and PEI (1 mg/mL) for the PEI-topped PEM surfaces (N=12). Once the first addition of positive polymer solution was added to each circle (N=36), they were left to incubate for 1 minute. After the incubation period, each individual surface was rinsed from top to bottom in order that the polymer was added (~1 second duration per surface) with a light stream (almost drop-wise) of DI water: the rinsing protocol is detailed in FIG. 5. With a single Petri lid held at an approximately 350 angle (a), three of six surfaces were rinsed individually from left to right (b) with the DI water waste collecting at the bottom lip of the lid. The lid was then rotated counterclockwise by 1800 (c), maintaining its angled position, allowing the excess water to flow around the lid edge. The final three surfaces were rinsed from above in the same order (left to right) as above (d) and the accumulated DI water was subsequently poured out (e). The lids were then flipped upside down and solidly tapped eight times against three piled Kimwipe on the lab bench removing excess DI water (f). This process was repeated for all remaining surfaces.

A small aliquot (100 μL) of PSS (1 mg/mL) was then added to all 36 surfaces in sequence and left to incubate for 1 minute. The same rinsing protocol (above) was used post incubation. This cycle of polymer addition and rinsing was repeated a total of 9 times for the (+) PEMs and 8 times for the (−) PEMs. The polymer incubation period for the last two layers of each PEM type (layers 10 and 11 of the (+) PEMs and layers 9 and 10 for the (−) PEMs) were left to incubate for 5 minutes. After the final rinsing step, when all polyelectrolyte layers had been added, the surfaces were left to dry with the dish lids partially off.

### Building the Fluidic Chip

Four strips of double-sided tape were cut (6 cm long) and one strip (i) was added between two of three PEM surfaces in each row (FIG. 4 b). On either side of the first strip of tape, two more strips (ii and iii) were added parallel to the first at 0.7 cm on either side. Finally, the last strip (iv) was added with the same 0.7 cm spacing to the left of strip iii. This was repeated for all six Petri dish lids.

Next, the top plastic layer of each strip of tape was carefully removed using a tweezer. Each PEM surface was topped with a thin glass coverslip (1.5×1.5 cm), where the PEM was positioned at the centre and the coverslip edges contacted the double-sided tape strips on either side (FIG. 4 c). Pressure was applied, by hand, at all points of contact between the coverslip and tape to maximize adhesion. All chip inlets and the inlet-facing edge of all coverslips had hydrophobic boundaries drawn on with a permanent marker (FIG. 4 c).

### Preparing Cell Suspensions

Bacteria were first prepared by inoculating the strain of interest and incubating it overnight with shaking (220 RPM) at 37° C. in LB. Bacteria were sub-cultured the next day 1:1000 into fresh LB and incubated with shaking at 37° C. between 4-6 hours to the desired optical densities (OD600 nm). Strains, SNPs, OD₆₀₀ measurements and cell suspension compositions can be found in Table 1. Staphylococcus aureus ATCC29213 waIR EV and waIR murOP were both grown in LB containing 12 μg/mL chloramphenicol and methicillin-resistant Staphylococcus aureus (MRSA) CA-USA300 ΔtarO-10 was grown in LB containing 300 μg/mL spectinomycin.

Next, two 1 mL aliquots were transferred to individual Eppendorf tubes and centrifuged at 8000×g for 3 minutes to obtain a pellet for each strain. The supernatant was carefully removed and the pellet was resuspended with 500 μL PBS. The 500 μL suspensions of two tubes from the same original culture were combined to produce a concentrated suspension (1 mL) each. The cells were further rinsed two more times with PBS. Subsequently, all PBS concentrated suspensions were further serially diluted two-fold to produce four unique dilutions per suspension labelled C1-C4 from lowest to highest cell concentration. All bacteria were tested using three biological replicates, where each suspension was prepared from a single colony.

Table 1: Table of bacteria tested and cell suspension optical densities.

A list of all bacterial species tested, their strains and SNPs, Gram stain, and liquid suspension optical densitites (OD₆₀₀) pre- and post-PBS resuspension. Acronyms CA, MRSA, VISA stand for community-associated, methicilin-resistant Staphylococcus aureus and vancomycin-intermediate Staphylococcus aureus, respectively.

| | | | Pre-Resuspension OD₆₀₀ | | | PBS Suspension OD₆₀₀ | | | | |
|---|---|---|---|---|---|---|---|---|---|---|
| | | Gram | Culture | Culture | Culture | Culture | Culture | Culture | | |
| Bacterial Species | Strain/SNP | Stain | A | B | C | A | B | C | Mean | SEM |
| Acinetobacter baumanii | B0098426R | negative | 0.494 | 0.562 | 0.519 | 0.736 | 0.788 | 0.734 | 0.753 | 0.018 |
| Klebsiella pneumoniae | H0142357 | negative | 0.477 | 0.491 | 0.488 | 0.744 | 0.719 | 0.707 | 0.723 | 0.011 |
| Pseudomonas aeruginosa | PA01 | negative | 0.452 | 0.498 | 0.468 | 0.653 | 0.720 | 0.710 | 0.694 | 0.021 |
| Enterococcus faecalis | ATCC29212 | positive | 0.296 | 0.300 | 0.298 | 0.767 | 0.702 | 0.726 | 0.732 | 0.019 |
| MRSA | CA-USA300-10 | positive | 0.535 | 0.486 | 0.514 | 0.770 | 0.725 | 0.718 | 0.738 | 0.016 |
| VISA | C506 | positive | 0.588 | 0.556 | 0.574 | 0.748 | 0.794 | 0.762 | 0.768 | 0.014 |
| Staphylococcus aureus | ATCC29213 | positive | 0.536 | 0.575 | 0.509 | 0.726 | 0.753 | 0.741 | 0.740 | 0.008 |
| Staphylococcus aureus | ATCC29213 walR | positive | 0.472 | 0.441 | 0.450 | 0.793 | 0.722 | 0.783 | 0.766 | 0.022 |
| Staphylococcus aureus | ATCC29213 walK | positive | 0.489 | 0.509 | 0.494 | 0.725 | 0.734 | 0.750 | 0.736 | 0.007 |
| Staphylococcus aureus | ATCC29213 walR EV | positive | 0.529 | 0.542 | 0.576 | 0.695 | 0.712 | 0.776 | 0.728 | 0.025 |
| Staphylococcus aureus | ATCC29213 walR murOP | positive | 0.478 | 0.468 | 0.439 | 0.734 | 0.766 | 0.697 | 0.732 | 0.020 |
| MRSA | CA-USA300 ΔtarO-10 | positive | 0.506 | 0.525 | 0.429 | 0.713 | 0.689 | 0.682 | 0.695 | 0.009 |
| Blind Testi (E. faecalis) | ATCC29212 | positive | 0.244 | 0.262 | 0.250 | 0.532 | 0.574 | 0.657 | 0.588 | 0.037 |
| Blind Testii (A. baumanii) | B0098426R | negative | 0.338 | 0.390 | 0.391 | 0.757 | 0.695 | 0.779 | 0.744 | 0.025 |
| Blind Testiii (K. pneumoniae) | H0142357 | negative | 0.420 | 0.398 | 0.419 | 0.739 | 0.675 | 0.703 | 0.706 | 0.019 |

### Facilitating Bacterial-PEM Interactions

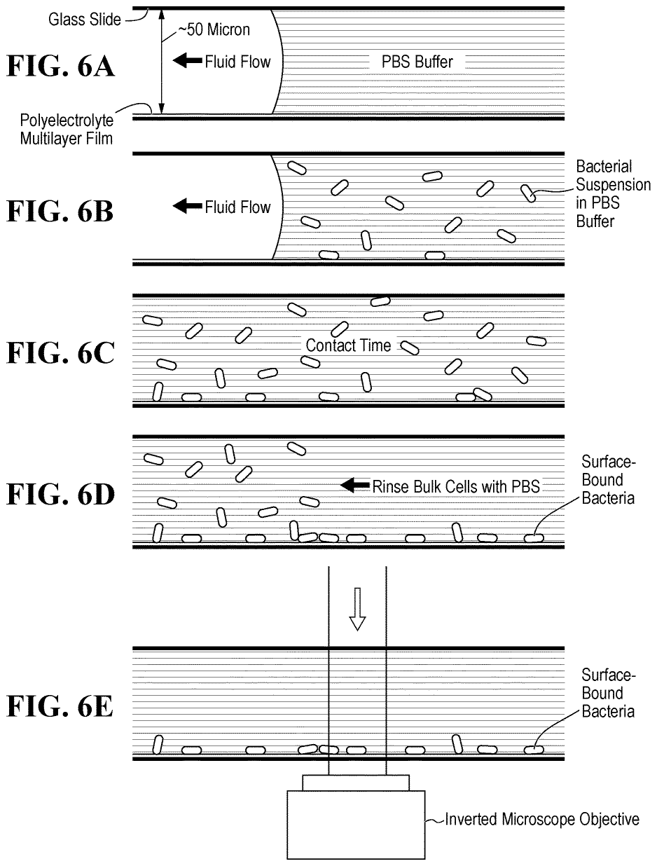

All plates were arranged by PEM type in a biosafety cabinet with Petri covers removed. Groups of three PEMs in a row were marked C1-C4 covering all cell suspension concentrations per PEM type (N=3 per concentration per PEM type). The steps involved in the addition of a given bacterial suspension to a fluid lane is illustrated in FIGS. 6 a-e. Once the same-day bacterial suspensions reached the desired range of optical densities, all 36 fluid lanes were pre-wet with 100 μL 1× phosphate buffer saline (PBS, pH=7.4) for 30 minutes (a). Post wetting, excess PBS buffer was carefully wiped off utilizing Kimwipes at both fluid lane inlets and outlets, without drying the lanes. To begin round one of cell-surface interactions, 70 μL of the C1 and C2 cell suspensions were pipetted onto the PDDA and PEI lane inlets (N=12), wicked through in sequence utilizing Kimwipes in contact with the outlet (b) and left to interact for 10 minutes (c). The same process above was applied for round two adding C3 and C4 cell suspensions to PDDA and PEI lane inlets (N=12) with the 10-minute incubation period starting at the 5-minute mark for round one. At the first net 10-minute mark, lanes from round one (N=12) were rinsed with a 100 μL aliquot of PBS buffer in sequence that the suspensions were wicked through (d). This step was repeated for a total of 200 μL PBS wicked through utilizing a small, roughly equivalently sized piece of dry Kimwipe per lane. The rinsing process was repeated at the net 15-minute mark for all round two lanes (N=12). Once completed, the addition of cell suspension, incubation (10 minutes), and rinsing protocols were repeated for all PSS lanes (N=12) covering all cell suspensions (C1-C4) at once in round three.

### Capturing Surface-Bound Cells and Image Processing

With all lanes rinsed, each PEM surface (N=36) was imaged using an inverted microscope 10 times per surface (N=360 total images per type of bacterium) (FIG. 6 e). All images were taken at 200× magnification and the positions along each surface were selected at random. Areas containing large pieces of dust or debris were avoided.

All images were batch processed in ImageJ to count the surface-bound cells: the left and right edges were cropped out followed by applying the FindMaxima function (15 prominence, excluding edge cases) to extract cell counts. The batch processing macros code is as follows:

makeRectangle(503, 1, 925, 1079);
run(“Crop”);
run(“Find Maxima...”, “prominence=15 exclude output=Count”);

### Building Attachment Curves and Surface Interaction Profiles (SIPs)

The mean cell counts across replicates per added bacterial suspension concentration (OD₆₀₀) were plotted to produce attachment curves (mean±SEM) per PEM type per bacterium. All cell counts across all replicates were plotted per added bacterial suspension concentration (OD₆₀₀) and a linear regression was applied. The interaction strength values (slope values) per bacterial-PEM combination were collected per bacterium to produce each bacteria-specific SIP.

### Example 1

Polyelectrolyte multilayer (PEM) films were prepared via layer-by-layer assembly (FIG. 1 a) whereby oppositely charged polymers were deposited in sequence to build a robust, uniform film. Three unique PEM films were prepared using a combination of three polyelectrolytes: the strongly positive polydiallyldimethylammonium chloride (PDDA) (FIG. 1 b), the strongly negative polystyrene sulfonate (PSS) (FIG. 1 c), and the weakly positive polyethylenimine (PEI) (FIG. 1 d).

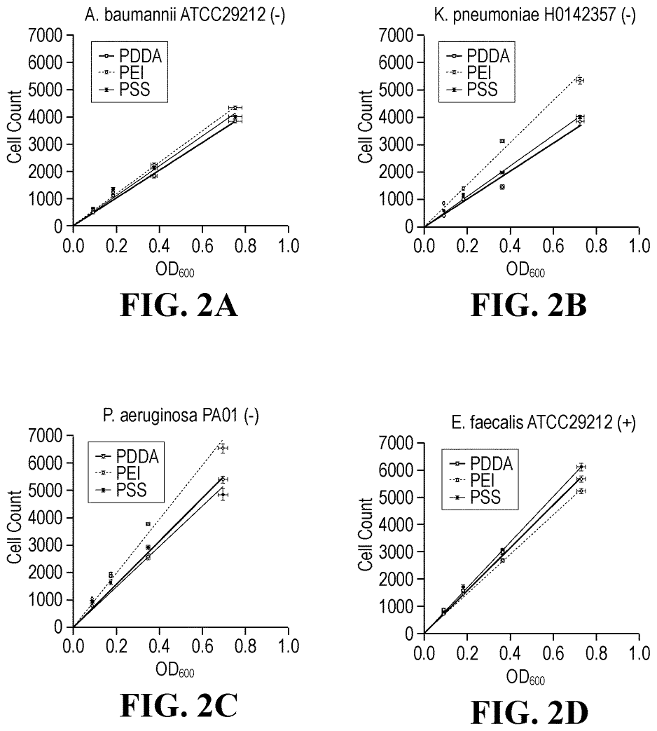

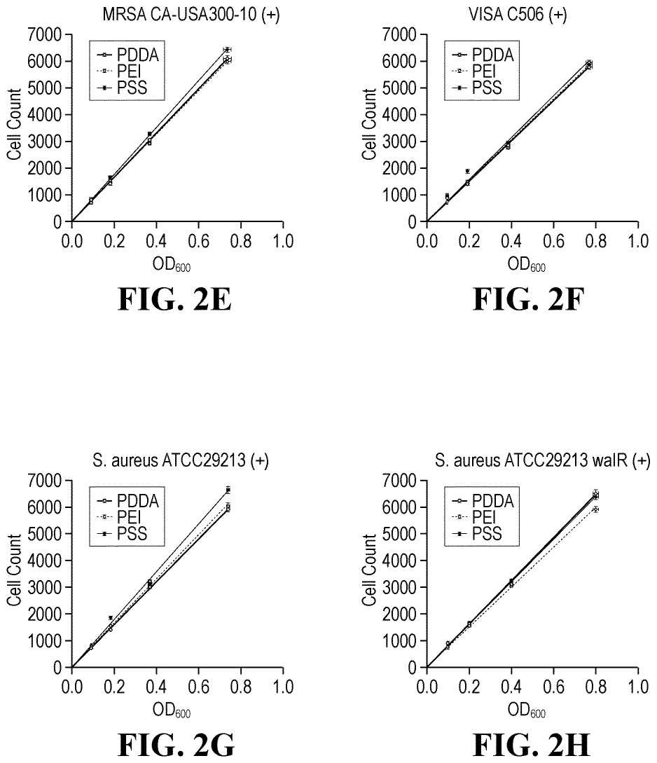

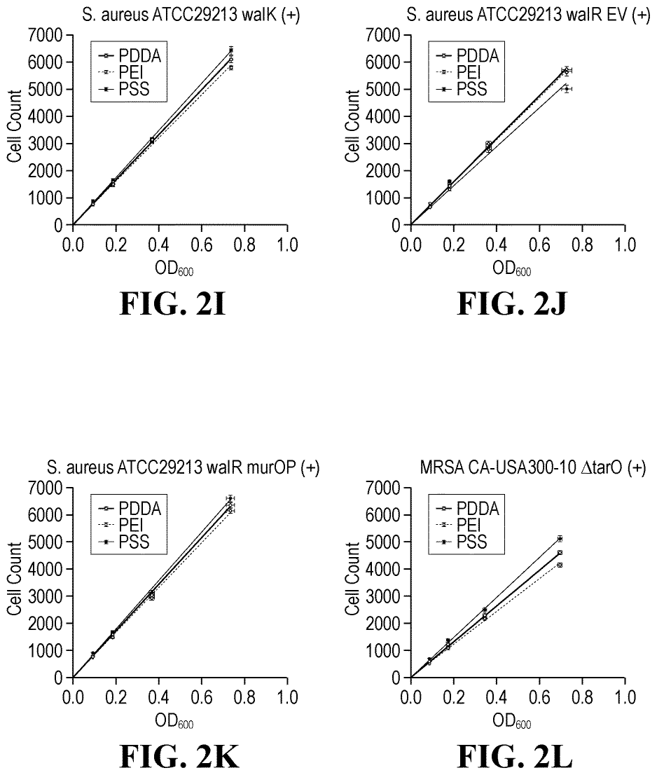

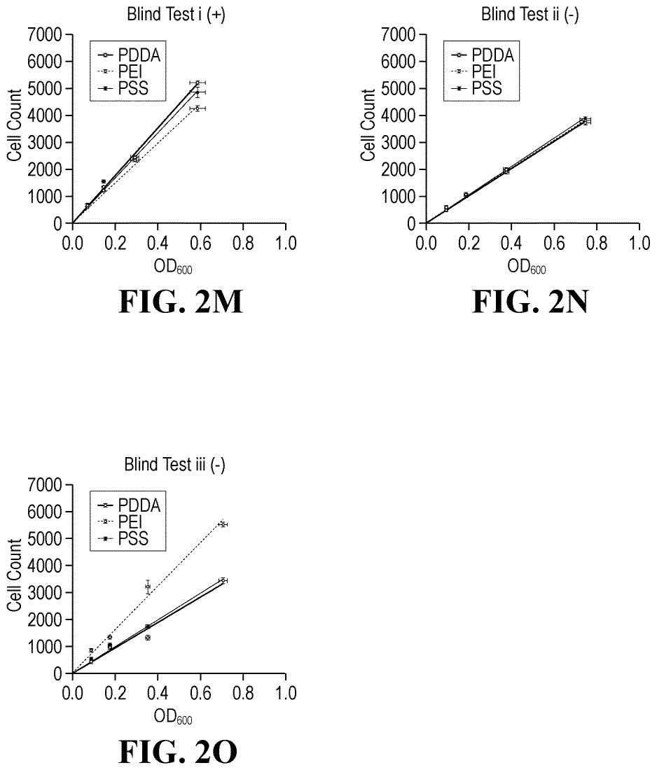

The prepared PEM films were encased in microfluidic channels (FIG. 1 e) with defined dimensions aimed at limiting the variability in fluid flow rate and direction. Sheer stress caused by fluid flow can impact bacterial attachment (see for example, Persat, A. et al. The Mechanical World of Bacteria. Cell 161, 988-997 (2015)), requiring a consistent control over the hydrodynamics of the channels. Cell suspensions at varying bulk concentrations were introduced to individual channels involving pre-soaking (1), the addition of a cell suspension (2), a 10 -minute incubatory period (3), and the removal of excess bulk cells via rinsing (4) (FIG. 1 f). The number of surface-bound bacteria was then quantified by microscopy and plotted against each respective cell suspension concentration (FIGS. 2 a-o). The slopes of these curves represented the strengths of interaction values collected to construct each unique SIP.

Surface interaction strengths of six nosocomial ESKAPE pathogens were plotted as a heat map (FIG. 1 h) and produced distinct SIPs that differed in their relative magnitudes of interaction strength per PEM. While grouping SIPs by Gram stain (FIG. 1 i) we observed a stronger attachment preference for PEI in Gram-negative ESKAPE pathogens, whereas the Gram-positive ESKAPEs preferred PSS. Differences in exposed surface chemistries between these two classes of bacteria are significant given their unique exterior compositions (see for example, Silhavy, T. J., Kahne, D. & Walker, S. The Bacterial Cell Envelope. Cold Spring Harb. Perspect. Biol. 2, a000414 (2010)). These differences appear more nuanced when compared to the level of individual species within these groups, where surface proteins, receptors, and molecules, including the dominant Gram-negative lipopolysaccharides (LPS) and Gram-positive teichoic acids (TA), display minor variations in their relative abundance and chemical compositions.

### Example 2

As further proof of concept, the SIP of a wild type (WT) methicillin-resistant S. aureus (MRSA USA300) was compared to a tarO deletion mutant that lacks teichoic acids (FIG. 1 j). Depleted of teichoic acids, it was observed that an altered SIP where all PEM surface interactions had been considerably reduced. It is notable that the degree of percent downshift for PEI compared with PDDA and PSS surfaces was considerably larger: PEI (25.2%), PDDA (19.3%), and PSS (15.8%) in the tarO deletion mutant. This could be suggestive of the repellent nature of an exposed peptidoglycan layer towards PEI that is otherwise buffered in the presence of TAs. Moreover, this may help to explain why in the Gram-negative cases, where the peptidoglycan is completely unexposed, PEI preference is comparatively much higher.

### Example 3

To challenge this method further, SIPs of different types of S. aureus having single nucleotide polymorphisms (SNPs) in the wal operon, a key regulator of the autolysin-encoding genes in cell wall metabolism (Dubrac, S., Boneca, I., Poupel, 0. & Msadek, T. New Insights into the WaIK/WaIR (YycG/YycF) Essential Signal Transduction Pathway Reveal a Major Role in Controlling Cell Wall Metabolism and Biofilm Formation in Staphylococcus aureus. J. Bacteriol. (2007) doi:10.1128/JB.00645-07) were generated and compared. The first case study is S. aureus with a single-point mutation in wal, with reduced peptidoglycan recycling, and a subsequent single-point mutation in walK which restores cell wall metabolism back to approximately wild-type levels. SIPs are distinguishable between these SNPs (FIG. 1 k), and while the magnitude of SIP from walR to walK reverts towards that of the WT, the order of preference of positive surfaces does not revert. These shifts are also echoed in a second case study whereby cell wall metabolism was restored instead through the addition of the mupGmurQmurP operon encoded on a plasmid (FIG. 1 l), again highlighting the ability of SIPs to distinguish between types of bacteria only differing by SNPs. To illustrate this further, this approach was applied to S. aureus WT, MRSA, and vancomycin-intermediate Staphylococcus aureus (VISA). Each SNP subsequently increases in its extent of antibiotic resistance and once again displays unique SIPs (FIG. 1 m).

### Example 4

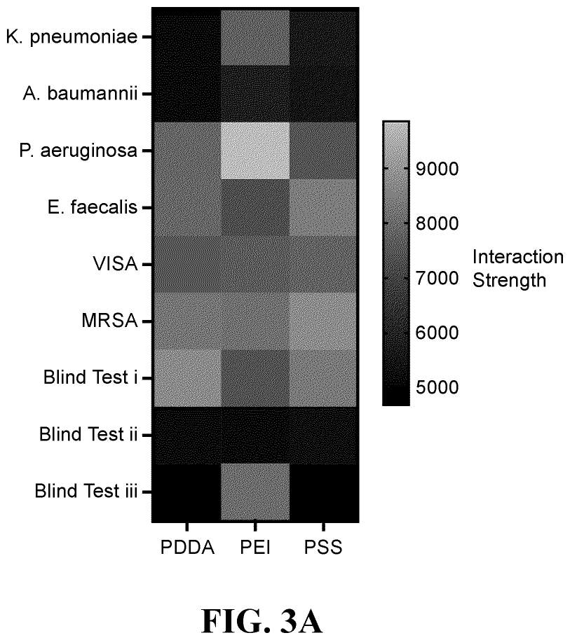

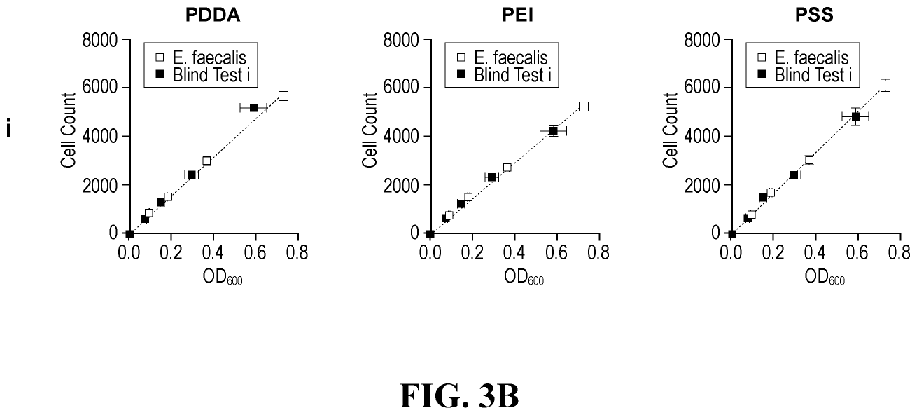

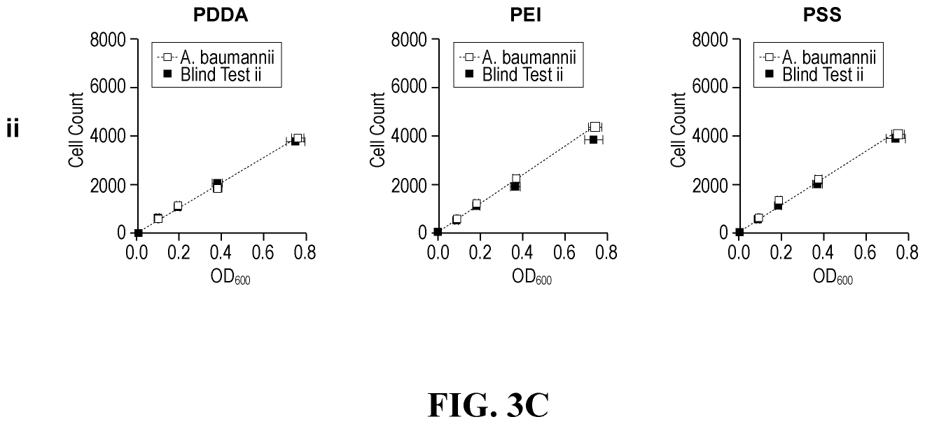

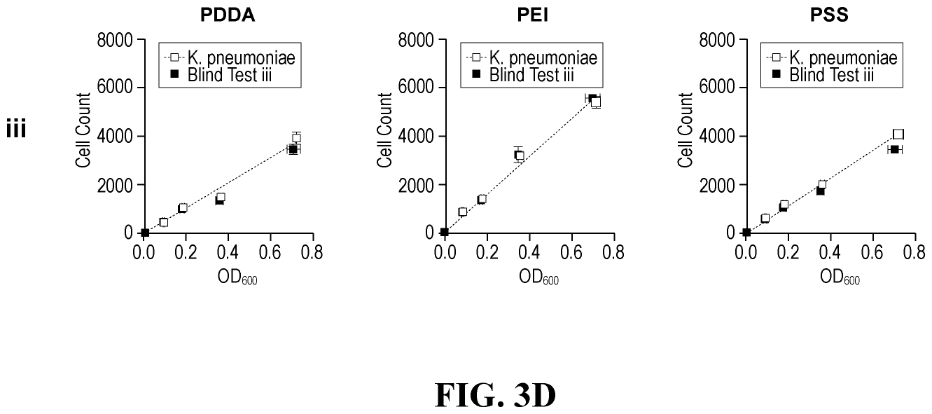

A set of three blind tests were completed whereby species of the six tested ESKAPE pathogens were selected at random. Using a minimum of three surface types to distinguish between them, each test pathogen was successfully identified in a blind experiment (FIG. 3 a): i) Enterococcus faecalis, ii) Acinetobacter baumannii, and iii) Klebsiella pneumoniae. A further breakdown of the percent agreement between attachment curve slopes of ESKAPE and blind test pathogens is detailed in Table 2. While there were some minor deviations in the interaction slopes (FIG. 3 b-d) in one of the three surfaces for each blind test, these had no significant comparison to other ESKAPE SIPs.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Furthermore, material appearing in the background section of the specification is not an admission that such material is prior art to the invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. A method of preparing a premeasurement for a known type of a bacteria, the method comprising:
a) providing a defined material;
b) contacting a fluid with the defined material for a first period of time, the fluid comprising the bacteria and having a known concentration, thereby forming a contacted material;
c) rinsing the contacted material with a rinsing solution by causing the rinsing solution to flow across the contacted material with a first generally consistent flow rate for a second period of time, thereby forming a rinsed contacted material;
d) measuring the bacteria on the rinsed contacted material, thereby generating a measurement; and
e) recording the measurement, thereby preparing a premeasurement for the known type of the bacteria.

2. A method of identifying a type of a bacteria, the method comprising:
a) providing a defined material;
b) contacting a fluid with the defined material for a first period of time, the fluid comprising the bacteria and having a known concentration, thereby forming a contacted material;
c) rinsing the contacted material with a rinsing solution by causing the rinsing solution to flow across the contacted material with a first generally consistent flow rate for a second period of time, thereby forming a rinsed contacted material;
d) measuring the bacteria on the rinsed contacted material, thereby generating a measurement; and
e) comparing the measurement to a premeasurement,
wherein the premeasurement is from a set of premeasurements, the set of premeasurements comprising a plurality of known measurements where each known measurement is a known measurement for one of a plurality of known types of bacteria and the known measurement is obtained using the same flow rates, the same periods of time, and the same defined material and a match between the measurement and the premeasurement identifies the type of the bacteria as the known type of bacteria from the matched premeasurement.

3. The method according to claim 2 wherein providing the defined material comprises prerinsing the defined material by contacting a prerinsing solution with the defined material.

4. The method according to claim 3 wherein contacting the prerinsing solution with the defined material comprises dipping the defined material in the prerinsing solution for a third period of time.

5. The method according to claim 3 wherein contacting the prerinsing solution with the defined material comprises adding the prerinsing solution to the surface of the defined material and allowing the prerinsing solution to sit for a third period of time.

6. The method according to claim 3 wherein contacting the prerinsing solution with the defined material comprises causing the prerinsing solution to flow across the surface of the defined material with a second generally consistent flow rate for a third period of time.

7. The method according to claim 6 wherein the second generally consistent flow rate is in a range of from about 0.0 cm/s to about 5 cm/s.

8.-10. (canceled)

11. The method according to claim 2 wherein the prerinsing the defined material by contacting a prerinsing solution with the defined material is repeated at least once.

12.-14. (canceled)

15. The method according to claim 2 wherein the method is repeated one or more times using a different defined material for each of the one or more times and wherein the different defined material is a substrate upon which a coating is deposited and the coating comprises a material selected from the group consisting of at least one of: a polymer, a polyelectrolyte, a nanoparticle, a surfactant: an organic molecule, a biomolecule, a protein, a poly amino acid, an oligonucleotide, and a nucleic acid.

16. (canceled)

17. The method according to claim 15 wherein the coating is a PEM and is deposited in one layer and the different defined material is PDDA, PEI, or PSS.

18. The method according to claim 15 wherein the coating is a PEM and is deposited in successive layers to form a multilayer film and the different defined material is PDDA, PEI, or PSS.

19. (canceled)

20. (canceled)

21. The method according to claim 2 wherein the defined material is a substrate upon which a coating is deposited and the coating comprises a material selected from the group consisting of at least one of: a polymer, a polyelectrolyte, a nanoparticle, a surfactant; an organic molecule, a biomolecule, a protein, a poly amino acid, an oligonucleotide, and a nucleic acid.

22. The method according to claim 21 wherein the coating is a PEM and the defined material is PDDA, PEI, or PSS.

23. (canceled)

24. The method according to claim 2 wherein the first generally consistent flow rate is in a range of from about 0.5 cm/s to about 5 cm/s.

25.-27. (canceled)

28. The method according to claim 2 wherein the first period of time is in a range of from about 1 minute to about 10 minutes.

29.-31. (canceled)

32. The method according to claim 2 wherein the contacting the fluid comprising the bacteria with the defined material for a first period of time, comprises causing the fluid comprising the bacteria to flow across the defined material with a third generally consistent flow rate for the first period of time.

33. The method of claim 32 wherein the third generally consistent flow rate is in a range of from about 0.05 cm/s to about 1 cm/s.

34. The method according to claim 2 wherein the set of premeasurements consists of a subset of premeasurements wherein the subset of premeasurements is limited to premeasurements of known types of bacteria of interest.

35. The method according to claim 2 wherein the measurement and each premeasurement is expressed as a member selected from the group consisting of: bacterial count per unit area, intensity of light transmitted through, intensity of light reflected, intensity of light diffracted, and intensity of light scattered.

36. The method according to claim 2 wherein the measurement is expressed as a bacterial count per unit area measurement and each premeasurement is expressed as a bacterial count per unit area premeasurement.

37. The method according to claim 2 wherein the premeasurement is expressed as an average obtained from averaging a plurality of premeasurements.
